(19) United States
(12) Patent Application Publication
(10) Pub. No.: US 2005/0096613 A1
(43) Pub. Date: May 5, 2005

(54) Cling film fastening system for disposable soft goods

(76) Inventors: James D. Carper, Waukesha, WI (US); Mark D. Alper, Mukwonago, WI (US)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 10/700,761

(22) Filed: Nov. 4, 2003

(51) Int. Cl. B32B 27/32; B32B 27/30; B32B 27/12; A61F 13/20; A61F 13/15; B32B 31/16; B32B 17/00; B27N 3/00; D21J 1/00; B31D 1/00; B32B 31/00; B31B 1/60; B65C 1/00; B65B 1/00

(52) U.S. Cl. 604/366; 604/393; 442/394; 442/396; 442/398; 156/60; 156/62.2; 156/73.1

(57) ABSTRACT

A unique laminate structure, a process for making the laminate, and use of the laminate in a fastening system for disposable soft goods is disclosed. The laminate is composed of a cling film layer having autoadhesive surface properties bonded to a flexible, but substantially non-stretchable, base carrier layer. The base carrier layer is formed of a material which eliminates, or substantially limits, any stretching of the cling film. The cling film layer includes a thermoplastic polymer coating that provides autoadhesive surface properties selected from the group consisting of polyolefins, acrylic modified polyolefins, vinyl acetate modified polyolefins and acrylic polymer. Preferably, the polyolefin may be polypropylene or polyethylene. Preferably, the carrier or base layer is comprised of a nonwoven material, or a thermoplastic film. A laminate composed of a cling film layer and a non-stretchable base layer is particularly useful as a fastening system for a disposable diaper or a feminine care pad with low peel, but high shear strength.

### BACKGROUND OF THE INVENTION

The present invention relates to fastening systems for disposable soft goods such as disposable diapers, and more particularly to a laminate composed of cling film and a non-stretchable substrate such as a non-woven, a process for making the cling film/nonwoven laminate, and various methods of using the cling film/nonwoven laminate such as, but not limited to, in a refastenable closure system for disposable soft goods.

The prior art is replete with numerous examples of constructions employed as a fastening system for disposable soft goods. Disposable soft goods refer to articles such as disposable diapers, sanitary napkins, surgical drapes, hospital gowns, hospital pads and many other utilitarian objects wherein one or more layer is composed of a nonwoven material. Examples of fastening systems for disposable soft goods can be found in numerous U.S. patents such as U.S. Pat. Nos. 4,973,326; 4,894,060; 4,726,971; 4,585,450; 4,540,414; 4,296,750; and 4,210,144.

Disposable diapers of the type widely used today generally comprise a three layer composite structure including a liquid permeable body side inner liner, a liquid impermeable outer layer or cover, and a batt of absorbent material sandwiched between the inner liner and outer cover. The diapers have front and rear panels joined together by a crotch section, and are typically made in an hourglass shape. The front and rear panels each have waist portions that encircle an infant's body and are overlapped and joined together to hold the diaper in place. In order to join the waist portions together, disposable diapers have included pressure sensitive adhesive tapes as fasteners for securing the front and rear panels together about the waist of an infant. Although pressure sensitive adhesive coated tapes have been used as the fasteners for disposable diapers, various other fastening systems have also been employed, such as combining the adhesive coated tape with a landing zone on the front panel of the diaper, as well as the use of hook and loop fasteners.

An inherent problem with the forgoing diaper fastening systems using pressure sensitive adhesive tabs is that of contamination of the tacky surfaces. Thus, talcum powder, baby oil or other foreign matter that finds its way onto either the pressure sensitive adhesive of the tab or onto the landing zone to which the pressure sensitive adhesive is adhered can reduce the reliability of the fastener, can limit the bonding strength of the adhesive, and/or can prevent fastening altogether. Although the use of hook and loop fasteners substantially overcomes the problem of reduced fastener reliability due to contaminants on pressure sensitive adhesive, hook and loop fastener systems are relatively expensive and may not be economical for use on inexpensive disposable diapers.

Disposable diapers utilizing tab fasteners which are coated with autoadhesives are also known in the art, as disclosed in U.S. Pat. No. 5,085,655. The tab fastener disclosed in U.S. Pat. No. 5,085,655 is in the form of a laminate having an autoadhesive layer formed by a thermoplastic elastomer and a base carrier layer formed by a layer of thermoplastic material. The autoadhesive surface is formed of a thermoplastic elastomer comprising a block copolymer having rubbery segments and non-rubbery segments. The thermoplastic carrier layer is preferably a polyolefin such as polyethylene or polypropylene, or a polyester. Tab fasteners of the construction disclosed in the '655 patent, however, suffer from the disadvantage of having relatively high peel strength. The examples given in the '655 patent show peel strength of 1364-2043 g/in. This is clearly too high to function as a workable refastenable tape tab. For example, when one attempts to remove fastening tabs of this type from a diaper after they have been secured in place, the autoadhesive layer will stretch or deform due to this relatively high peel strength resulting in the possibility of permanent deformation of the tab and the inability to refasten the tab if desired. This, for example, prevents a person from checking the diaper after it has been on an infant for a period of time, and then refastening the diaper if it has not been soiled. Thus, it would be desirable to provide a laminate structure which eliminates, or at least severely limits stretching and/or deformation of the autoadhesive layer.

### SUMMARY OF THE INVENTION

The present invention is directed toward a unique laminate structure, a process for making the laminate, and use of the laminate in various applications, preferably as a fastening system for disposable soft goods such as disposable diapers. The laminate is composed of a “cling” film layer having autoadhesive surface properties at ambient temperature bonded to a flexible, but substantially non-stretchable, base carrier layer.

The cling film layer is of the type such as those traditionally used as pallet stretch wrap. When used as pallet stretch wrap, the autoadhesive face of the cling film will engage the non-autoadhesive back of the cling film as it is wrapped about the pallet. However, in this invention, cling film is used in a non-traditional way, i.e. face to face rather than face to back.

The base carrier layer is formed of a material which eliminates, or substantially limits, any stretching of the cling film. The base carrier layer thus provides dimensional stability both in the longitudinal and/or the cross direction to prevent stretching or deformation of the cling film. Preferably, the laminate will stretch no more than about 50% in either direction, more preferably stretching should be limited to no more than about 25% from its original non-stretched configuration, and most preferably stretching in either direction should be less than 10%. Stretching and/or substantial deformation of the cling film layer is undesirable as it reduces the ability of the autoadhesive surface of the cling film to adhere to itself.

The laminates of the present invention are particularly useful in fastening systems for soft goods, especially disposable diapers. The laminates of the present invention are particularly useful in systems of the type including fastening tabs on the rear panel and a landing zone located on the front panel of the diaper. The laminates of the present invention may be used to provide both the fastening tabs and the reinforcing landing zone on the diapers. In such an application, the autoadhesive properties of the cling film layer eliminate or substantially reduce the contamination problems of the prior art systems using pressure sensitive adhesives. In addition, the autoadhesive surfaces of the cling film layer are substantially non-adhesive at ambient temperature with respect to the carrier or base layer thus enabling the laminate to be manufactured in the form of a web or roll for use in conventional diaper manufacturing systems. Also, a laminate composed of cling film layer and a non-stretchable base carrier layer provide the fastening system with low peel, but high shear strength. Preferably, the peel strength is 1000 g/inch or less, more preferably 600 g/inch or less, and most preferably 400 g/inch or less. On the other hand, the shear strength is preferably greater than 4 hours, and most preferably greater than 8 hours, as further described herein. Thus, the fastening tabs may be readily opened by a user without rupturing or significantly damaging the front panel and/or the landing zone of the disposable diaper while at the same time preventing stretching or deformation of the cling film layer itself so that the tabs may be refastened if desired.

The cling film layer is typically a laminate composed of a coating comprising a thermoplastic polymer material providing autoadhesive surface properties bonded to a structural substrate such as a polyolefin e.g. polyethylene. It is the structural substrate of the cling film layer which is bonded to the non-stretchable base carrier layer to form the desired laminate of the present invention. In some circumstances, however, the polymer coating can be directly bonded onto the base carrier layer to form the desired laminate of the present invention without the structural substrate component. For example, in this second embodiment, the cling film layer could be directly coextruded onto the nonwoven. Alternately, in a third embodiment, the base layer could be directly formed onto the cling layer. For example, if the base layer is a nonwoven material, the nonwoven can be directly melt blown onto the cling film layer.

The cling film substrate is composed of a thermoplastic polymer material selected from the group consisting of polyolefins, acrylic modified polyolefins, vinyl acetate modified polyolefins and acrylic polymers. Preferably, the polyolefin may be polypropylene or polyethylene. The acrylic modified polyolefin is preferably a copolymer or terpolymer of polypropylene or polyethylene and an acrylic. Likewise, the preferred vinyl acetate modified polyolefin is a copolymer or terpolymer of polypropylene or polyethylene and vinyl acetate.

The materials useful for forming the base carrier layer is any material which is flexible, but substantially non-stretchable in the machine direction (longitudinally) and/or the cross machine direction (transverse). Preferably, the carrier or base layer is comprised of a nonwoven material, which gives the laminate a soft, cloth-like feel, or a thermoplastic film such as a polyolefin.

The base layer and/or cling film layer can also be made breathable by any method known in the art.

### BRIEF DESCRIPTION OF THE DRAWINGS

The drawings illustrate the best mode presently contemplated of carrying out the invention.

In the drawings:

FIG. 1 is a front perspective view of a disposable diaper incorporating a first embodiment of the cling film/nonwoven laminate fastening system of the present invention;

FIG. 1A is a cross-sectional view of a cling film/non-woven laminate in accordance with the present invention;

FIG. 2 is a perspective view illustrating a portion of a diaper showing the initial closure made with the fastening system of FIG. 1;

FIG. 3 is a cross-sectional view taken through the plane of the line 3-3 in FIG. 2;

FIG. 4 is a front perspective view of a disposable diaper incorporating a second embodiment of a cling film/nonwoven laminate fastening system of the present invention;

FIG. 5 is a perspective view of a portion of a diaper showing the initial closure made with the fastening system of FIG. 4;

FIG. 6 is a cross-sectional view taken along the plane of the line 6-6 in FIG. 5;

FIG. 7 is a front perspective view of a disposable diaper incorporating a third embodiment of a cling film/nonwoven laminate fastening system of the present invention;

FIG. 8 is a perspective view of a portion of a diaper showing the initial closure made with the fastening system of FIG. 7;

FIG. 9 is a cross-sectional view taken along the plane of the line 9-9 in FIG. 8;

FIG. 10 is a front perspective view of a disposable diaper incorporating a fourth embodiment of a cling film/nonwoven laminate fastening system of the present invention;

FIG. 11 is a perspective view of a portion of a diaper showing the initial closure made with the fastening system of FIG. 10;

FIG. 12 is a cross-sectional view taken along the plane of the line 12-12 in FIG. 11;

FIG. 13 is a front perspective view of a disposable diaper incorporating a fifth embodiment of a cling film/non-woven laminate fastening system of the present invention;

FIG. 14 is a front perspective view of a disposable diaper incorporating a sixth embodiment of a cling film/non-woven laminate fastening system of the present invention;

FIG. 15 is a front perspective view of a disposable diaper incorporating a seventh embodiment of a cling film/non-woven laminate fastening system of the present invention;

FIG. 16 is a front perspective view of a disposable diaper incorporating an eighth embodiment of a cling film/non-woven laminate fastening system of the present invention;

FIG. 17 is a plan view of an absorbent article comprising a feminine care pad incorporating a cling film/non-woven laminate fastening system of the present invention; and

FIG. 18 is a perspective view of the feminine care pad of FIG. 17 shown as it would be worn by a user.

### DETAILED DESCRIPTION OF THE INVENTION

Referring now to the drawings, there is illustrated various embodiments of a laminate including integrally joined layers of a cling film having an outer surface with autoadhesive properties at ambient temperature mounted to a flexible, but substantially non-stretchable, base carrier substrate. The laminates of the present invention are useful as various components of nonwoven soft goods, and are particularly useful in fastening systems for soft goods, especially absorbent articles such as disposable diapers and feminine napkins used to absorb and contain exudates such as urine, discharged from a person's body. A laminate composed of cling film and a non-stretchable base carrier layer provides a fastening system with low peel, but high shear strength. In such applications, the autoadhesive properties of the outer surface of the cling film layer eliminate or substantially reduce the contamination problems of the prior art systems using pressure sensitive adhesives. In addition, the low peel, high shear properties of the laminate of the present invention eliminate or substantially reduce the deformation problems of prior art systems using an autoadhesive film layer such as those described in U.S. Pat. No. 5,085,655 (block copolymer based).

The material useful for forming the base carrier layer is any material which is flexible, but substantially non-stretchable in the machine direction (longitudinally) and/or cross machine direction (transverse). Preferably, the carrier base layer is comprised of a non-woven substrate or a thermoplastic film material. By “non-woven material” it is meant a sheet or web structure bonded together by entangling fiber or filaments (and by perforating films) mechanically, thermally or chemically. They are flat, porous sheets that are made directly from separate fibers or from molten plastic or plastic film. They are not made by weaving or knitting and do not require converting the fibers to yarn.

The thermoplastic film materials useful for forming the base carrier layer of the co-joined laminate include meltable film-forming thermoplastics which substantially do not adhere to the cling film autoadhesive surface at ambient temperature or service temperatures. Preferably, the thermoplastic film should have a melt temperature sufficiently close to that of the cling film substrate to enable co-extrusion of materials and formation of a permanent melt bond therebetween, with or without the use of an adhesive tie coat, which is retained after cooling. In practice, any thermoplastic film material may be used which is capable of being formed into a self-supporting continuous sheet or film having adequate mechanical properties to withstand normal handling and to fulfil the requirements of the end use application including satisfactory bonding with the cling film substrate at an elevated temperature, and to form a flexible, but substantially non-stretchable film. Thus, the base carrier layer is formed of a material which eliminates, or substantially limits, any stretching of the cling film. The base carrier layer thus provides dimensional stability both in the longitudinal as well as the cross-wise or transverse direction to prevent stretching or deformation of the cling film. The term “substantially non-stretchable” means that preferably, the base carrier layer will stretch no more than about 50% in either direction, and more preferably stretching should be limited to no more than 25% from its original non-stretched configuration, and most preferably less than 10% from its original non-stretched configuration. When bonded to the cling film layer, the base carrier layer will thus result in the laminate itself stretching during normal use no more than about 50% in either direction, and more preferably stretching no more than about 25%, and most preferably less than 10% from its original non-stretched configuration. Additionally, the base layer can be made breathable by any method known in the art.

The thermoplastic film material forming the base carrier layer may comprise a wide range of polymers, copolymers, terpolymers, interpolymers and blends thereof selected to meet the end use application. Illustrative thermoplastics which may be used alone or in blends include polyolefins such as polyethylene, polypropylene and polybutylene, copolymers of ethylene and C₃-C₈ olefins, thermoplastic polyesters, polyamides such as nylon, polysulfones, acrylic polymers such as polyethylene acrylic acid, polyethylene ethyl acrylate, polyethylene n-butyl acrylate and polyethylene methyl acrylate, polystyrene, polyurethanes, polycarbonates, halogenated polymers such as polyvinylchloride and polyvinylidene chloride, cellulosics, polyacrylonitriles, and ionomers based on sodium or zinc salts of ethylene/methacrylic acid. The preferred thermoplastic film materials comprise polyolefins including low, medium and high density polyethylene and, most preferably, polypropylene.

The cling film layer is typically a laminate composed of a coating comprising a thermoplastic polymer material providing autoadhesive surface properties bonded to a structural substrate such as a polyolefin, e.g. polyethylene or polypropylene. It is the structural substrate of the cling film layer that is in turn bonded to the non-stretchable base carrier layer to form the desired laminate of the present invention. In addition, the cling film layer may include one or more other substrates either between the structural substrate and the thermoplastic polymer coating, or on the side of the structural substrate that is opposite the thermoplastic polymer coating. For example, depending upon the tackiness of the autoadhesive surface, the structural substrate may need a release or slip additive layer thereon to prevent blocking when the cling film is stored in roll form. It should also be noted that in an alternate form the polymer coating that provides the autoadhesive surface properties for the cling film layer may be directly bonded (as for example by coextrusion) onto the base carrier to form the desired laminate with or without any other layer such as the structural substrate component. Additionally, the cling layer can be made breathable by any method known in the art.

The terms “autoadhesive” and “autoadhesion” and. “cling” are used herein to indicate the self-adhesive or co-adhesive-adhesive properties of a polymeric material which enable films, layers or coatings thereof to be repeatedly adhered together by application of pressure at service temperatures or room temperatures and separated. Such materials adhesively bond to each other but are substantially non-adhesive with respect to any other materials. The term “service temperature” is used herein in accordance with its ordinary meaning to indicate the intended temperature or temperature range of use for the autoadhesive cling film by the end user and/or under storage conditions of the end product. Thus, the service temperature typically ranges from ambient or room temperature of about 60° F. to a storage temperature of about 140° F.

The cling film substrate having the autoadhesive surface coating thereon is preferably composed of a thermoplastic material selected from the group consisting of polyolefins, acrylic modified polyolefins, vinyl acetate modified polyolefins, and acylic polymers. Preferably, the polyolefin may be polypropylene or polyethylene. The acrylic modified polyolefin is preferably a copolymer of polypropylene or polyethylene and an acrylic. Likewise, the preferred vinyl acetate modified polyolefin is a copolymer of polypropylene or polyethylene and vinyl acetate.

Additionally, small amounts, less than 25% by weight and more preferably less than 10% by weight of modifiers can be added to modify the auto-adhesive or other characteristics of the cling layer. Examples of these include tackifying resins, plasticizers, waxes, fillers, antioxidants, colorants, antiblocking agents, antistatic agents, UV stabilizers, etc.

The autoadhesive surface coating of the cling film substrate provides an adhesive surface that has low peel strength, but high shear strength. By “low” peel strength, it is meant that the peel strength is preferably 1000 g/inch or less, more preferably 600 g/inch or less, and most preferably 400 g/inch or less. By “high” shear strength, it is meant that the shear strength of the cling film is preferably greater than 4 hours, and most preferably greater than 8 hours as determined by the shear strength test hereinafter described. Thus, the low peel, but high shear strengths of the cling film autoadhesive surface enables the fastening tab of a disposable diaper to be readily opened by a user without rupturing or significantly damaging the front panel of the disposable diaper while at the same time allowing the tabs to be refastened if desired. Three examples of cling films that may be useful in the present laminate can be found in U.S. Pat. No. 5,049,423, U.S. Pat. No. 5,085,927 and U.S. Pat. No. 5,902,684. Other examples of cling film can be found in U.S. Pat. Nos. 5,093,188 and 5,208,096. The preferred cling film useful in the present laminate is a polyethylene film available under the trade name “Presto” from Presto Products Co. of Appleton, Wis. Two grades particularly well suited are Presto CNC 10152 and 101515. Another preferred polyethylene cling film is available under the trade name “Paragon” from Paragon Films, Inc. of Broken Arrow, Okla. Examples from Paragon include V109015A, T128370 Global and T817125. Yet another preferred cling film is an ethylene-alkyl acrylate available under the trade name “Pactiv APM3-2015” from Pactiv Corporation of Lake Forest, Ill. “Masking” films are also available from Tredegar Co.

The laminate of the present invention may be prepared by co-extrusion processing of the cling film layer directly onto the base carrier layer using any conventional commercially available apparatus. Alternately, the cling film may be adhesively bonded to the carrier base layer. Any other bonding method may be used to bond the cling film to the base layer, e.g. ultrasonic, thermal, pressure bonding, microwave, RF, etc. Typically, the adhesive layer may be any suitable hot melt adhesive, and may be applied using any standard application equipment either to the structural substrate of the cling film layer or to the base carrier layer or to both. Typical add-on levels for the adhesive layer would be from about 1 g/sq. meter to about 20 g/sq. meter. It should be noted that the particular apparatus selected, whether it be a co-extrusion apparatus or a coater/lamination apparatus, may depend upon the differences in processing temperatures and rheologies of the materials forming the cling film layer, the base carrier layer, and the optional adhesive layer.

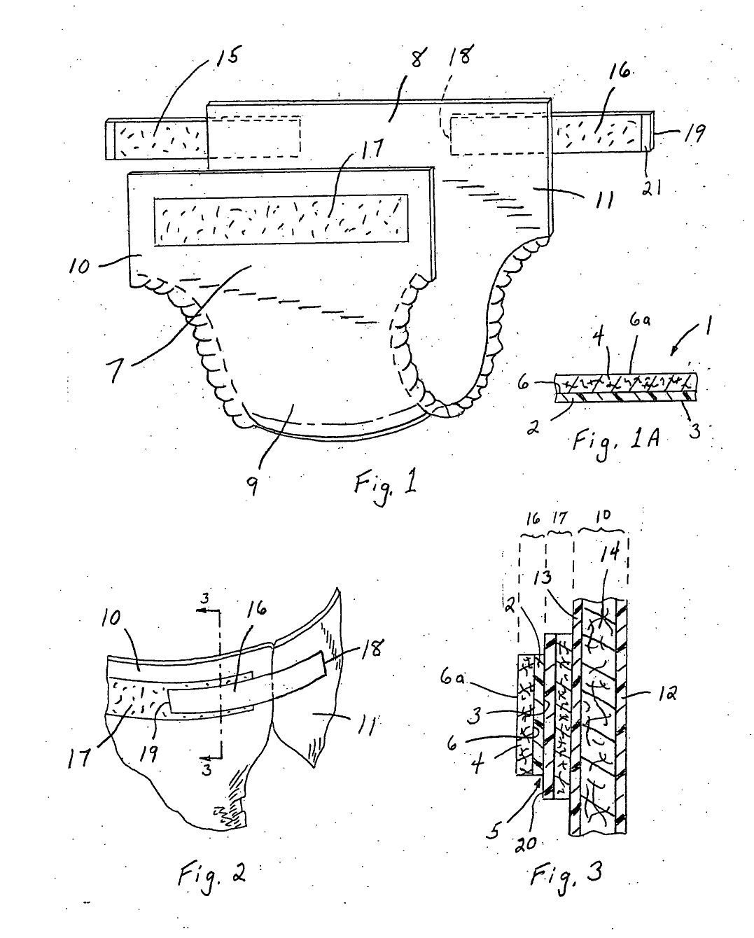

Referring now to FIGS. 1-3, and particularly to FIG. 1A, a laminate 1 of multi-layer construction includes a cling film 2 having an autoadhesive surface 3 bonded to and integrally joined with a flexible, but substantially non-stretchable, base carrier layer 4. The cling film 2 and base carrier layer 4 are bonded together along an interface 6 formed by the adjacent interior surfaces of film 2 and layer 4. As illustrated, cling film 2 is co-extruded directly onto base carrier layer 4. Both the cling film 2 and the base carrier layer 4 may range in thickness from about 0.1 mil to about 20 mils.

The cling film 2 is formed of a suitable thermoplastic polymer material, such as polyethylene. The exterior surface 3 of film 2 is autoadhesive in that it mutually adheres to like autoadhesive surfaces, but is otherwise substantially non-adhesive. The base carrier layer 4 is formed of a non-woven material and includes exterior surface 6a which does not adhere to surface 3 when they are pressed together at room temperature or elevated storage temperature conditions, even when wound under tension in large diameter rolls. Accordingly, the laminate 1 may be self-wound or stacked without a release liner thus enabling the laminate to be manufactured in the form of a web or roll for use in conventional diaper manufacturing systems.

As shown in FIGS. 1-3, laminate 1 is illustrated as being useful in a fastening system for a disposable diaper. As illustrated, the disposable diaper generally comprises a front panel 7 and a rear panel 8 joined together by a crotch section 9. The front panel 7 and rear panel 8 each have waist portions 10 and 11 respectively and encircle an infant's body, and are overlapped and joined together by the diaper fastening system to hold the diaper in place. The disposable diaper itself comprises a three-layer composite structure including a liquid permeable body side inner liner or top sheet 12, a liquid impermeable outer layer or back sheet 13, and a batt or core 14 of absorbent material sandwiched between the inner liner 12 and outer cover 13. As illustrated, a pair of diaper fastening tabs 15 and 16 each incorporating laminate 1 as its principal component is illustrated. Laminate 1 may be sold in roll form to a diaper manufacturer for die cutting to form tabs 15 and 16. The tabs 15 and 16 are secured to the outer liner 13 of the disposable diaper in a conventional construction.

As further illustrated in FIGS. 1-3, a fastening tab landing zone 17 incorporating laminate 1 as its principal component is secured to outer liner 13 along front panel 7. Landing zone 17 is conventionally adhesively bonded to the outer surface of cover 13, although other bonding means can be used. An additional piece of laminate (not shown) can be placed in a separate area to facilitate closure after the diaper is soiled for disposal purposes, if desired.

As shown best in FIG. 1, tabs 15 and 16 each has an elongate rectangular shape including an inner end 18 attached to waist portion 11 of rear panel 8 and an outer end 19. The base carrier layer 4 of laminate 1 at the terminal edge of outer end 19 extends slightly beyond the cling film layer 2 at the terminal edge of outer end 19 in the storage position and during use of the diaper to provide a finger lift area 21. No release liner or protective tab is required along the finger lift portion of outer end 19 since autoadhesive surface 3 is substantially non-adhesive and non-tacky. For purposes of diaper closure, the autoadhesive surface 3 of outer end 19 of fastening tabs 15 and 16 are pressed against the autoadhesive surface 20 of landing zone 17 forming a cling-to-cling interface 5. As noted earlier, tabs 15 and 16, as well as landing zone 17 is formed using laminate 1, and the overall configuration is illustrated in section in FIG. 3. Thus, the autoadhesive surface 3 of fastening tabs 15 and 16 are pressed against the autoadhesive surface 20 of landing zone 17. Thus, tabs 15 and 16 may be “fastened” to landing zone 17 resulting in waist portions 10 and 11 joined together to hold the diaper in place.

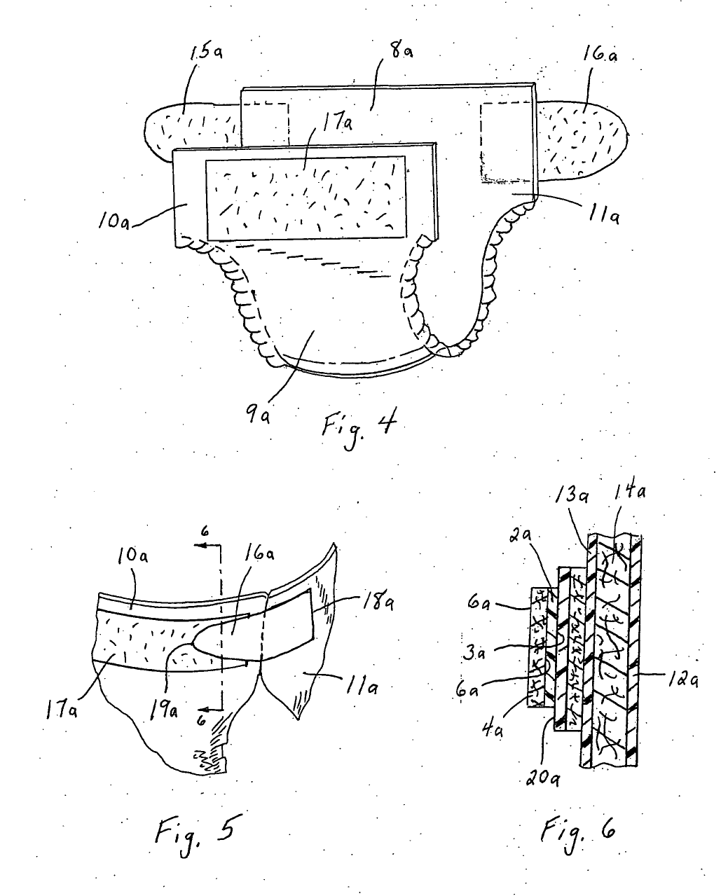

FIGS. 4-6 illustrate a disposable diaper incorporating a second embodiment of the cling film/non-woven laminate fastening system of the present invention. In this second embodiment, like components are numbered similarly as the first embodiment except using the subscript “a”. As illustrated, the only significant difference between this second embodiment of a diaper fastening system and the first embodiment shown in FIGS. 1-3, is that landing zone 17a is slightly larger than landing zone 17 shown in FIG. 1, and fastening tabs 15a and 16a are in the shape of ears instead of conventional rectangular shaped tapes. In all other aspects, the second embodiment of FIGS. 4-6 is the same as the first embodiment of FIGS. 1-3.

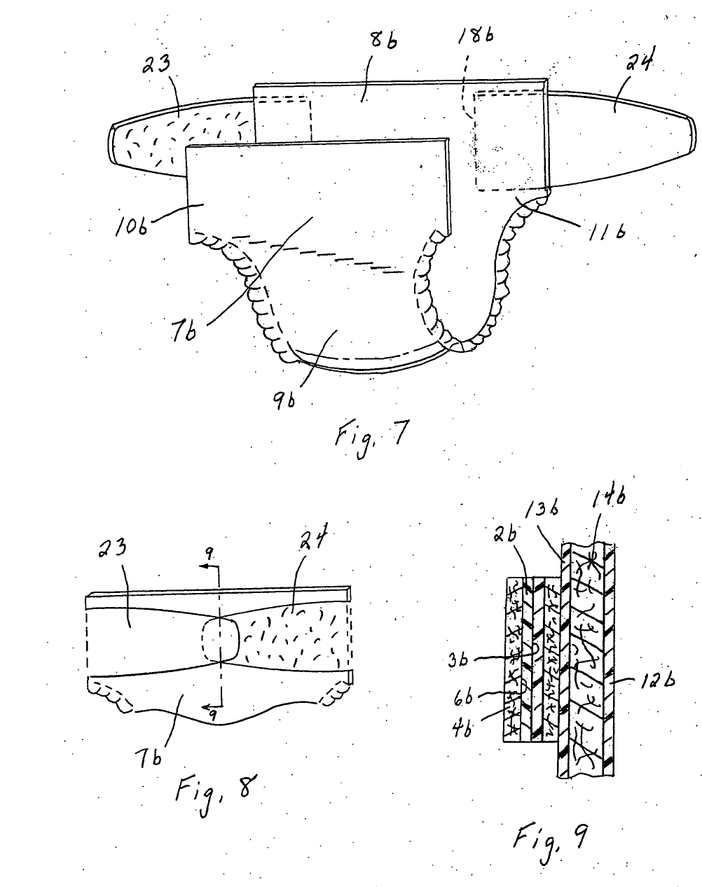

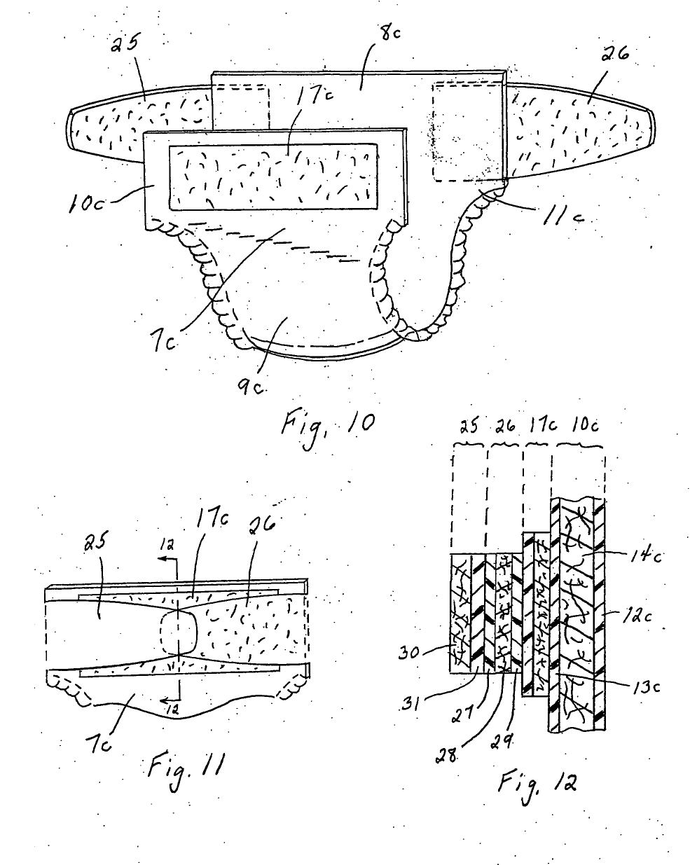

Referring now to FIGS. 7-9, there is illustrated a disposable diaper incorporating a third embodiment of the cling film/non-woven laminate fastening system of the present invention. In this third embodiment, like components are numbered similarly as the first and second embodiments except using the subscript “b”. This third embodiment eliminates the use of a landing zone on the front panel of the diaper. Instead, it incorporates a pair of large ears 23 and 24 with the autoadhesive surface of the cling film on one ear 23 facing up and the autoadhesive surface of the cling film of ear 24 facing down such that the outer edges of ears 23 and 24 overlap to provide a diaper fastening or closure system.

FIGS. 10-12 illustrate a disposable diaper incorporating a fourth embodiment of the cling film/non-woven laminate fastening system of the present invention. In this fourth embodiment, like components are numbered similarly as the first through third embodiments except utilizing the subscript “c”. This fourth embodiment is similar to the third embodiment of FIGS. 7-9 except it utilizes a landing zone 17c on the front panel 7c of the diaper and a pair of large ears 25 and 26 both composed of laminate 1. However, in this fourth embodiment, ear 26 has an autoadhesive surface on both sides thereof and thus forms a tri-laminate comprised of a cling film 27, a base carrier layer 28 and a second cling film 29 as shown best in FIG. 12. Ear 25, however, is similar to ears 15a and 16a in that it has a base carrier layer 30 and a cling film 31 on only one side thereof. As a result, when the fastening system is closed, the inner ear 26 clings to landing zone 17c and the outer 25 clings to a portion of landing zone 17c as well as a portion of the outer edge of ear 26.

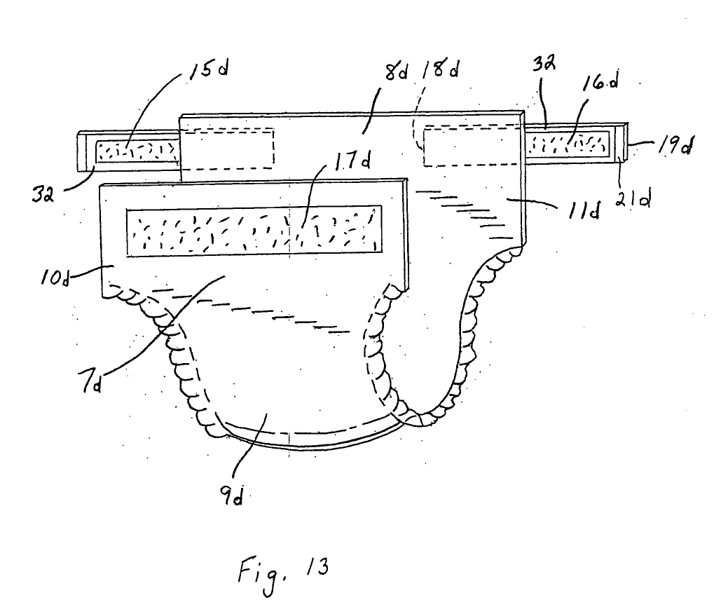

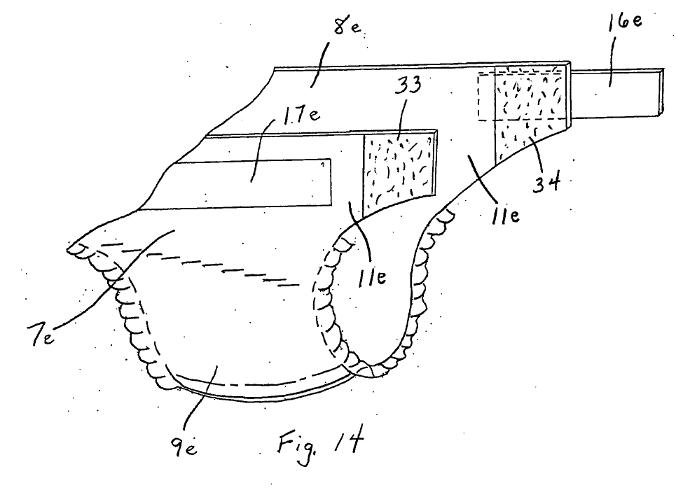

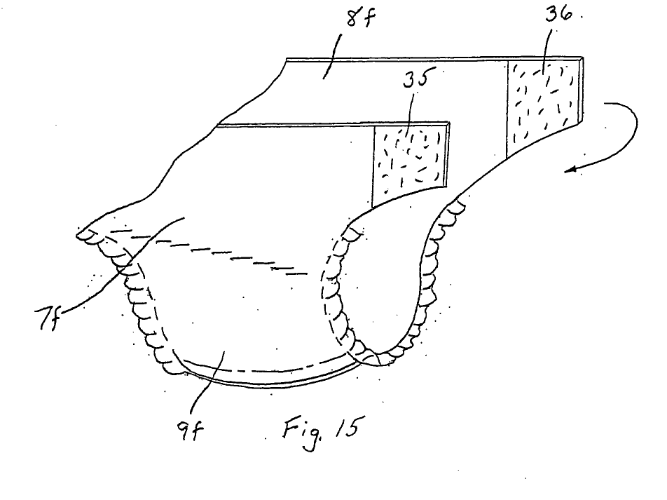

Referring now to FIG. 13, there is illustrated a disposable diaper incorporating a fifth embodiment of the cling film/non-woven laminate fastening system of the present invention. In this fifth embodiment, like components are numbered similarly as the first through fourth embodiments except utilizing the subscript “d”. This fifth embodiment is similar to the first embodiment except it utilizes an area 32 along the edges of fastening tabs 15d and 16d which contains a pressure-sensitive adhesive coated thereon. The pressure-sensitive adhesive area 32 may be utilized as a supplemental or secondary closure feature to ensure that the edge margins of tabs 15d and 16d are affixed to landing zone 17d. The pressure-sensitive adhesive area 32 thus ensures that the edges of tabs 15d and 16d do not curl up during use. Any pressure-sensitive adhesive commonly used in the prior art can be utilized to coat area 32, if desired.

FIG. 14 illustrates a disposable diaper incorporating a sixth embodiment of the cling film/non-woven laminate fastening system of the present invention. In this sixth embodiment, like components are numbered similarly as the first through fifth embodiments except utilizing the subscript “e”. This sixth embodiment utilizes a landing zone 17e on the front panel 7e of the diaper and a fastening tab 16e coated with pressure-sensitive adhesive or Velcro or other mechanical fastener. However, in the embodiment shown in FIG. 14, the waist portions 10e (not shown) and 11e of the diaper include ears 33 and 34 bonded along the sides thereof. The ears 33 and 34 are composed of the cling film/non-woven laminate described herein. In use, the outwardly facing cling surface of ear 33 overlaps the inwardly facing cling surface of ear 34 to provide a closure system, and the tab 16e engages landing zone 17e to provide a supplemental fastening system for a disposable diaper.

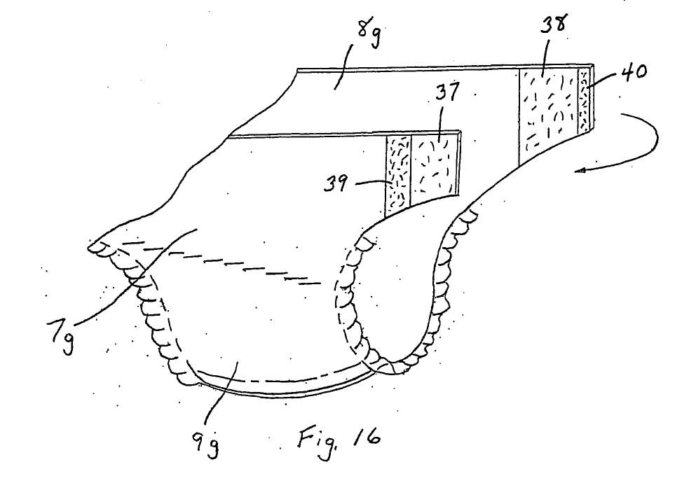

FIG. 15 illustrates a disposable diaper incorporating a seventh embodiment of the cling film/non-woven laminate fastening system of the present invention. In this seventh embodiment, like components are numbered similarly as the first through sixth embodiments except using the subscript “f”. This seventh embodiment is similar to the sixth embodiment of FIG. 14 except it eliminates the fastening tab 16e and landing zone 17e shown in FIG. 14. Instead, this embodiment incorporates only the integral ears 35 and 36 to function as the fastening system for a disposable diaper. As illustrated, the autoadhesive surface of the cling film of ear 35 faces outwardly while the autoadhesive surface of ear 36 faces inwardly so that when overlapped, the ears 35 and 36 provide a diaper fastening or closure system.

It should be noted that instead of adhesively attaching ears 35 and 36 to waist portions 10f and 11f of the diaper, ears 35 and 36 could also be formed integrally as part of the back sheet or outer liner 12f for the disposable diaper. In other words, FIG. 3 illustrates the diaper as including an inner liner or top sheet 12, an outer liner or back sheet 13, and an inner batt or absorbent core 14 sandwiched therebetween. It is contemplated that laminate 1 could replace the outer liner or back sheet 13 in such a manner that the non-woven based carrier layer would face outwardly and the cling film layer would face inwardly against batt 14. In this manner, ears 35 and 36 could be formed integrally as part of that outer laminate forming the back sheet. Thus, in the embodiment shown in FIG. 15, the ears 35 and 36 could be integrally formed with laminate 1 as a replacement for back sheet 12 instead of adhesively attached to the sides of waist portions 10f and 11f.

FIG. 16 illustrates a disposable diaper incorporating an eighth embodiment of the cling film/non-woven laminate fastening system of the present invention. In this eighth embodiment, like components are numbered similarly as the first through seventh embodiments except utilizing the subscript “g”. This eighth embodiment is similar to the seventh embodiment except it utilizes a hook and loop fastening system as a supplement to the cling film/non-woven laminate. As illustrated, this eighth embodiment includes a pair of ears 37 and 38 wherein ear 37 has an autoadhesive surface facing outwardly and ear 38 has an autoadhesive surface facing inwardly. However, in addition, ear 37 includes a strip 39 of hook fastening material and ear 38 includes a strip 40 of loop fastening material. Thus, when ears 37 and 38 are positioned in overlapping relation, the autoadhesive surfaces cling to one another to form a closure system and the strips 39 and 40 provide a supplemental closure to ensure a more secure diaper fastening system. It should be noted that the fastening material of strips 39 and 40 could be reversed, i.e. strip 40 could be the hook fastener while strip 39 could be the loop fastener, if desired. Also, the specific location and/or configuration of strips 39 and 40 could vary depending upon the desired amount of supplemental closure desired.

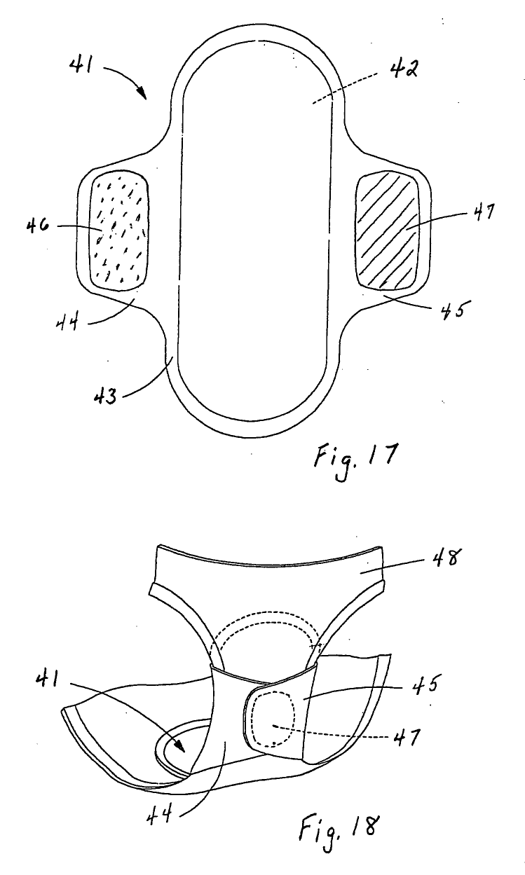

In a further alternative embodiment, the cling film/non-woven laminate may serve as a fastening system for a feminine care pad or sanitary napkin 41 as shown in FIGS. 17-18. The sanitary napkin 41 has a top sheet 42, a back sheet 43 and wings 44 and 45. As shown, the cling film/non-woven laminate may form the back sheet 43, the wings 44, 45, or both. Preferably, the cling film/non-woven laminate is disposed on wings 44 and 45 as illustrated by areas 46 and 47 respectively. Alternately, the entire wing 44 and/or 45 could be composed of the cling film/non-woven laminate. It should be noted that the autoadhesive surface of area 46 faces upwardly in FIG. 17 while the autoadhesive surface of area 47 faces downwardly in the paper (as illustrated by the cross hatching) in FIG. 17. As illustrated in FIG. 18, the cling film/non-woven laminates 46, 47 act to connect the wings 44 and 45 to each other around the wearer's underwear 48.

### EXAMPLE 1

The improved peel and shear properties of laminates made in accordance with the present invention are illustrated by the data reported in Table 1 below. For each laminate tested the peel strength was 50 g or less and shear strength was acceptable (all samples held for at least 8 hours), not only at room temperature but also after aging at elevated temperatures.

TABLE 1

| | | | | Stored 24 Hours | | Stored 24 hours | | |
|---|---|---|---|---|---|---|---|---|
| | | Initial Test | | at 120° F. | | at 100° F. | | |
| | | Room | | Room | | Room | | |
| | | Temp. | | Temp. | | Temp. | | |
| | | Peel | | Peel | | Peel | | |
| | | Strength | 100° F. | Strength | 100° F. | Strength | 100° F. | Chemistry of |
| Cling Film | NW | (gm) | Shear | (gm) | Shear | (gm) | Shear | Cling Surface |
| Pactiv APM3-2015 | Avgol | 50 | pass | 58 | pass | 34 | pass | Poly(Ethylene- |
| | | | | | | | | alkyl acrylate) |
| Presto CNC 101515 | Avgol | 30 | pass | 22 | pass | 18 | pass | Polyethylene |
| Presto CNC 10152 | Avgol | 13 | pass | 20 | pass | 19 | pass | Polyethylene |
| Paragon V1 09015A | Avgol | 32 | pass | 19 | pass | 14 | pass | Polyethylene |
| Paragon T1 28370 Global | Avgol | 17 | pass | 24 | pass | 22 | pass | Polyethylene |
| Paragon T8 17125 | Avgol | 16 | pass | 16 | pass | 15 | pass | Polyethylene |

Method for Preparing the Cling Laminates:

The cling films were laminated using a Nordson coater/laminator. A pressure sensitive hot melt adhesive was used at an add-on level of 10 grams per square meter and was applied using standard meltblown application equipment. The adhesive was applied to the film substrate and nipped to the non-cling side of the cling film after an open time of 250 milliseconds. After bonding the two substrates together, the resultant cling laminate was wound onto itself. The adhesive used to prepare the laminates was H2545 and is available from Bostik Findley, Inc. The nonwoven is a standard spunbond polypropylene nonwoven with a basis weight of 14 gsm available from Avgol Nonwoven Industries.

Peel test Method:

The peel test was performed using an Instron tensile tester with a crosshead speed of 36 inches/minute. A two inch wide sample of laminate was placed with the cling side to the cling side of a second laminate of the same width. A 500 gram roller was used to compress the laminates before testing. The test method used was a standard 180 degree peel test. The average peel strength in grams is reported in the tables. Duplicates were also tested after they were stored in an incubator oven for a period of 24 hours at 100° F. and 120° F. The laminates were not bonded during the elevated temperature storage. After aging the samples were tested as before.

Shear Test Method:

A two inch wide sample of cling laminate was placed in contact with a second laminate with the cling sides touching. The overlap area was two inches by 1 1/2 inches. A standard 500 gram roller was used to compress the structure. A 500 gram weight was used to stress the bonded area in a modified 180° shear configuration while in an incubator oven at 100° F., i.e. the shear sample was placed around a 6 inch core member with the bonded area at about the 9 o'clock position. If the bond held for a period of four hours, it was considered to have passed the test.

Pactiv APM 3-2015 stretch film is available from Pactiv Corporation, 1900 West Field Court, Lake Forest, Ill. 60045.

Presto films are available from Presto Products Company, P.O. Box 2399, Appleton, Wis. 54912.

Paragon films are available from Paragon Films, Inc., 3500 West Tacoma, Broken Arrow, Okla. 74012.

Depending upon the end use requirements of the fastening system on the finished article, the desired peel strength could be higher than that described in Example 1, i.e. up to 1000 g/inch.

### EXAMPLE 2

This example was performed to determine the effect of aging on the peel strength of cling laminates constructed in accordance with the present invention, and to compare the data obtained with that of prior art laminates disclosed in U.S. Pat. No. 5,085,655. Accordingly, the peel test method described in Example 1 was once again performed on two inch wide samples of laminate except using a crosshead speed of 10 inches/minute. The average peel strength in grams is reported in Table 2A initially, after one hour, after one day and after 13 days for laminates made in accordance with the present invention. All samples were stored at room temperature (RT) for the designated time period. These data are then compared to the results reported in Mann et al U.S. Pat. No. 5,085,655 which describes a prior art laminate using a styrene-ethylene-butylene-styrene (SEBS) block copolymer or an ethylene-propylene rubber (EPR) as an autoadhesive layer. The Mann et al data is reported in Table 2B.

TABLE 2A

| | 10″/min @RT | | | |
|---|---|---|---|---|
| | Initial | | | |
| Film | (gm) | 1 Hour (gm) | 1 Day (gm) | 13 Days (gm) |
| Pactiv 2015 | 50 | 19 | 13 | 17 |
| Presto CNC 101515 | 30 | 20 | 22 | 13 |
| Presto CNC 10152 | 13 | 18 | 20 | 20 |
| V1 09015A | 32 | 16 | 14 | 14 |
| T1 28370 Global | 17 | 18 | 17 | 20 |
| T8 17125 | 16 | 21 | 19 | 20 |

TABLE 2B
(From U.S. Pat. No. 5,085,655)

| | 10″/min @RT | | | |
|---|---|---|---|---|
| Film | Initial (gm) | 1 Hour (gm) | 1 Day (gm) | 13 Days (gm) |
| SEBS/SEBS | N/A | 1364-2043 | 1364-2043 | 2272-2725 |
| (Kraton | | | | |
| 1657) | | | | |
| EPR/EPR | N/A | 1590-2271 | 1818-2735 | 2725-3179 |
| (Vistalon | | | | |
| 719) | | | | |

One can conclude from the above data that the peel strengths of the cling laminates of the present invention tested do not increase to any significant degree during aging, even after 13 days of aging. In contrast, the peel strengths of the prior art laminates increased dramatically over time.

### EXAMPLE 3

As a further comparison, the peel and shear properties of several currently available commercial diaper fastening systems were obtained and tested in the same manner as for Example 1. The data is illustrated in Table 3 below. It is to be noted that the peel strength for the tape fastening systems tested are significantly higher than the laminates of the present invention and increase substantially during aging for 8 hours at 100° F.

Peel Values for Various Commercial Fastening Systems:

Several samples of commercially sold diapers were obtained for testing. Two of them used a conventional pressure sensitive tape tab and two used a mechanical fastener system. The same basic peel test that was described previously was used with the following modifications.

The landing zone for each diaper was cut out of the diaper. In the case of the pressure sensitive tape tab, the landing zone consisted of a piece of polypropylene film bonded to the backsheet with a release coating on an outward side. The pressure sensitive tape tab was placed on the release side of the structure and rolled down with a 500 gram roller. In the case of the mechanical fastener, the landing zone consisted of a “loop” material bonded to the backsheet of the diaper. The “hook” tab portion was placed in contact with the “loop” side of the landing zone and rolled down with a 500 gram roller. A 180 degree peel test was performed using the same conditions as noted before. The average peel strength was noted. The pressure sensitive tape tabs were also aged while bonded for eight hours at 100° F. to see if the peel strength changed. The mechanical fasteners were not aged since the bonds should be unaffected by aging.

TABLE 3

| | Brand | Peel Strength | Type |
|---|---|---|---|
| Sample One - Initial | Toys “R” Us | 181 grams | Tape |
| Sample One - Aged | Toys “R” Us | 393 grams | Tape |
| Sample Two - Initial | Amostra | 260 grams | Tape |
| Sample Two - Aged | Amostra | 827 grams | Tape |
| Sample Three | Huggies Supreme | 42 grams | Mechanical |
| | | | Fastener |
| Sample Four | Pampers Swaddlers | 89 grams | Mechanical |
| | | | Fastener |

Shear tests were also run in accordance with the method set forth in Example 1 on Samples 1 and 2 of Example 3. All samples passed the four hour test initially. No shear testing was performed on the aged samples. However, it is expected that the aged samples would pass.

Although shear testing was not performed on Samples 3 and 4 of Example 3, it is expected that they would pass this test.

1. A laminate structure comprising:
a base layer comprised of a flexible, substantially non-stretchable substrate having a first bonding side; and
a cling film layer bonded to the first bonding side of said base layer to form a laminate, said laminate having a peel strength of 1000 g/inch or less and a shear strength greater than 4 hours.

2. The laminate of claim 1 wherein said base layer is selected from the group consisting of a nonwoven, and a thermoplastic film selected from the group consisting of polyolefins such a polyethylene, polypropylene and polybutylene, copolymers of ethylene and C₃-C₈ olefins, polyesters, polyamides such as nylon, polysulfones, acrylic polymers such as polyethylene methyl acrylic acid, polyethylene-n-butyl acrylate, polyethylene ethyl acrylate and polyethylene methyl acrylate, polystyrene, polyurethanes, polycarbonates, halogenated polymers such as polyvinylchloride and polyvinylidene chloride, cellulosics, polyacrylonitriles, and ionomers based on sodium or zinc salts of ethylene/methacrylic acid.

3. The laminate of claim 1 wherein said base layer is a nonwoven.

4. The laminate of claim 1 wherein said cling film layer includes a thermoplastic polymer coating selected from the group consisting of a polyolefin, an acrylic modified polyolefin, a vinyl acetate modified polyolefin, and an acrylic polymer.

5. The laminate of claim 4 wherein said acrylic modified polyolefin is a copolymer of a polyolefin and acrylic.

6. The laminate of claim 4 wherein said vinyl acetate modified polyolefin is a copolymer of a polyolefin and vinyl acetate.

7. The laminate of claim 4 wherein said polyolefin is polypropylene.

8. The laminate of claim 4 wherein said polyolefin is polyethylene.

9. The laminate of claim 1 wherein said laminate stretches less than about 50% from its original non-stretched configuration.

10. The laminate of claim 1 wherein said base layer is breathable.

11. The laminate of claim 1 wherein said cling film layer is breathable.

12. An absorbent article used to absorb and contain body exudates, the absorbent article comprising:
a top sheet;
a back sheet joined with at least a portion of the top sheet;
an absorbent core disposed between at least a portion of the top sheet and the back sheet; and
a fastening system for fastening the absorbent article in a usable position, said fastening system includes a laminate structure comprising a base layer having a flexible substantially non-stretchable substrate with a first bonding side, and a cling film layer bonded to the first bonding side of said base layer to form said laminate structure, said laminate structure having a peel strength of 1000 g/inch or less and a shear strength greater than 4 hours.

13. The absorbent article of claim 12, wherein said base layer is selected from the group consisting of a nonwoven, and a thermoplastic film selected from the group consisting of polyolefins such a polyethylene, polypropylene and polybutylene, copolymers of ethylene and C₃-C₈ olefins, polyesters, polyamides such as nylon, polysulfones, acrylic polymers such as polyethylene methyl acrylic acid, polyethylthyl acrylate and polyethylene methyl acrylate, polystyrene, polyethylene-n-butyl acrylate, polyurethanes, polycarbonates, halogenated polymers such as polyvinylchloride and polyvinylidene chloride, cellulosics, polyacrylonitriles, and ionomers based on sodium or zinc salts of ethylene/methacrylic acid.

14. The absorbent article of claim 12 wherein said base layer is a nonwoven.

15. The absorbent article of claim 12 wherein said cling film layer is a thermoplastic polymer coating selected from the group consisting of a polyolefin, an acrylic modified polyolefin, a vinyl acetate modified polyolefin, and an acrylic polymer.

16. The absorbent article of claim 15 wherein said acrylic modified polyolefin is a copolymer of a polyolefin and acrylic.

17. The absorbent article of claim 15 wherein said vinyl acetate modified polyolefin is a copolymer of a polyolefin and vinyl acetate.

18. The absorbent article of claim 15 wherein said polyolefin is polypropylene.

19. The absorbent article of claim 15 wherein said polyolefin is polyethylene.

20. The absorbent article of claim 12 wherein said laminate stretches less than about 50% from its original non-stretched configuration.

21. The absorbent article of claim 12 wherein said base layer is breathable.

22. The absorbent article of claim 12 wherein said cling film layer is breathable.

23. The absorbent article of claim 12 wherein said laminate structure is breathable.

24. The absorbent article of claim 12 wherein said base layer is pigmented.

25. The absorbent article of claim 12 wherein said cling film layer is pigmented.

26. The absorbent article of claim 12 wherein said laminate structure is pigmented.

27. The absorbent article of claim 12 wherein said fastening system is part of a disposable diaper.

28. The absorbent article of claim 27 wherein said disposable diaper has a first waist portion, a second waist portion, a crotch section located between the first and second waist portions, and said fastening system is configured to join said first and second waist portions when the diaper is configured in said usable position.

29. The absorbent article of claim 28 wherein said laminate structure is selected from the group consisting of an ear and a tab on at least one of said waist portions.

30. The absorbent article of claim 29 wherein said laminate structure is bonded to said at least one waist portion.

31. The absorbent article of claim 29 wherein said laminate structure is integrally formed as part of the back sheet of said diaper.

32. The absorbent article of claim 28 wherein said disposable diaper further includes a landing zone, said landing zone comprised of said laminate structure.

33. The absorbent article of claim 28 wherein said disposable diaper further includes a landing zone, said landing zone comprised of said cling film layer.

34. The absorbent article of claim 12 wherein said fastening system is part of a feminine napkin.

35. The absorbent article of claim 34 wherein said feminine napkin includes a pair of wings extending laterally outwardly in opposite directions from the absorbent core, and said fastening system is configured to join said wings when the feminine napkin is configured in said usable position.

36. The absorbent article of claim 35 wherein said laminate structure is bonded to said wings.

37. The absorbent article of claim 35 wherein said laminate structure is integrally formed as part of the back sheet of said feminine napkin.

38. The absorbent article of claim 35 wherein said wings comprise said laminate structure.

39. A method of manufacturing an absorbent article having at least one fastening member for fastening said absorbent article in a usable position, comprising the steps of
selecting for said at least one fastening member a laminate structure, said laminate structure comprising
a base layer comprised of a flexible, substantially non-stretchable substrate having a first bonding side;
a cling film layer bonded to the first bonding side of said base layer to form a laminate, said laminate having a peel strength of 1000 g/inch or less and a shear strength greater than 4 hours; and
applying said fastening member to the absorbent article.

40. The method of claim 39 wherein said base layer is selected from the group consisting of a nonwoven, and a thermoplastic film selected from the group consisting of polyolefins such a polyethylene, polypropylene and polybutylene, copolymers of ethylene and C₃-C₈ olefins, polyesters, polyamides such as nylon, polysulfones, acrylic polymers such as polyethylene methyl acrylic acid, polyethylene-n-butyl acrylate, polyethylene ethyl acrylate and polyethylene methyl acrylate, polystyrene, polyurethanes, polycarbonates, halogenated polymers such as polyvinylchloride and polyvinylidene chloride, cellulosics, polyacrylonitriles, and ionomers based on sodium or zinc salts of ethylene/methacrylic acid.

41. The method of claim 39 wherein said base layer is a nonwoven.

42. The method of claim 39 wherein said cling film layer is a thermoplastic polymer coating selected from the group consisting of a polyolefin, an acrylic modified polyolefin, a vinyl acetate modified polyolefin, and an acrylic polymer.

43. The method of claim 42 wherein said acrylic modified polyolefin is a copolymer of a polyolefin and acrylic.

44. The method of claim 42 wherein said vinyl acetate modified polyolefin is a copolymer of a polyolefin and vinyl acetate.

45. The method of claim 42 wherein said polyolefin is polypropylene.

46. The method of claim 42 wherein said polyolefin is polyethylene.

47. The method of claim 39 wherein said laminate stretches less than about 50% from its original non-stretched configuration.

48. The method of claim 39 including the step of forming a landing zone on said absorbent article comprised of said laminate structure.

49. The method of claim 39 including the step of forming a landing zone on said absorbent article comprised of said cling film layer.

50. A method of manufacturing a laminate structure, including the steps of:
providing a base layer comprised of a flexible, substantially non-stretchable substrate having a first bonding side;
providing a cling film layer comprised of a thermoplastic polymer material that provides an autoadhesive surface; and
combining said base layer and cling film layer together to form a laminate.

51. The method of claim 50 wherein said base layer is selected from the group consisting of a nonwoven, and a thermoplastic film selected from the group consisting of polyolefins such a polyethylene, polypropylene and polybutylene, copolymers of ethylene and C₃-C₈ olefins, polyesters, polyamides such as nylon, polysulfones, acrylic polymers such as polyethylene methyl acrylic acid, polyethylene-n-butyl acrylate, polyethylene ethyl acrylate and polyethylene methyl acrylate, polystyrene, polyurethanes, polycarbonates, halogenated polymers such as polyvinylchloride and polyvinylidene chloride, cellulosics, polyacrylonitriles, and ionomers based on sodium or zinc salts of ethylene/methacrylic acid.

52. The method of claim 50 wherein said base layer is a nonwoven.

53. The method of claim 50 wherein said cling film layer is a thermoplastic polymer coating selected from the group consisting of a polyolefin, an acrylic modified polyolefin, a vinyl acetate modified polyolefin, and an acrylic polymer.

54. The method of claim 53 wherein said acrylic modified polyolefin is a copolymer of a polyolefin and acrylic.

55. The method of claim 53 wherein said vinyl acetate modified polyolefin is a copolymer of a polyolefin and vinyl acetate.

56. The method of claim 53 wherein said polyolefin is polypropylene.

57. The method of claim 53 wherein said polyolefin is polyethylene.

58. The method of claim 50 wherein said laminate stretches less than about 50% from its original non-stretched configuration.

59. The method of claim 50 wherein the step of combining comprises extruding the cling film layer onto the bonding side of said base layer.

60. The method of claim 50 wherein said base layer is a nonwoven material, and the step of combining comprises melt blowing the nonwoven material onto the cling film layer.

61. The method of claim 50 wherein the step of combining comprises adhesively bonding the cling film layer to the bonding side of said base layer.

62. The method of claim 50 wherein the step of combining comprises thermally bonding the cling film layer to the bonding side of said base layer.

63. The method of claim 50 wherein the step of combining comprises ultrasonically bonding the cling film layer to the bonding side of said base layer.

64. The method of claim 50 wherein the step of combining comprises RF bonding the cling film layer to the bonding side of said base layer.

65. The method of claim 50 wherein the step of combining comprises microwave bonding the cling film layer to the bonding side of said base layer.

66. The method of claim 50 wherein the step of combining comprises pressure bonding the cling film layer to the bonding side of said base layer.
